(19) United States
(12) Patent Application Publication
(10) Pub. No.: US 2022/0202429 A1
(43) Pub. Date: Jun. 30, 2022

(54) CLOT RETRIEVAL DEVICES AND THERAPEUTIC USE THEREOF

(71) Applicants: BALT EXTRUSION, MONTMORENCY (FR); INSTITUT NATIONAL DE LA SANTE ET DE LA RECHERCHE MEDICALE (INSERM), PARIS (FR); UNIVERSITE PARIS 13 - PARIS NORD, VILLETANEUSE (FR); UNIVERSITE DE PARIS, PARIS (FR); FONDATION A DE ROTHSCHILD, PARIS (FR)

(72) Inventors: Jean-Philippe Mickael DESILLES, PARIS (FR); Mikael MAZIGHI, MAISONS LAFFITTE (FR); Giuseppina CALIGIURI, PARIS (FR); Antonino NICOLETTI, PARIS (FR)

(73) Assignees: BALT EXTRUSION, MONTMORENCY (FR); INSTITUT NATIONAL DE LA SANTE ET DE LA RECHERCHE MEDICALE (INSERM), PARIS (FR); UNIVERSITE PARIS 13 - PARIS NORD, VILLETANEUSE (FR); UNIVERSITE DE PARIS, PARIS (FR); FONDATION A DE ROTHSCHILD, PARIS (FR)

(21) Appl. No.: 17/564,617

(22) Filed: Dec. 29, 2021

(30) Foreign Application Priority Data
Dec. 29, 2020 (EP) 20306701.2

(51) Int. Cl. A61B 17/221; A61F 2/95; C08K 5/3415; C09D 179/02; A61L 31/10

(52) U.S. Cl. CPC A61B 17/221; A61F 2/95; A61B 2017/00526; C09D 179/02; A61L 31/10; C08K 5/3415

(57) ABSTRACT

The present invention relates to improved clot retrieval devices, wherein at least the part of the device intended to be in contact with a clot is coated with at least one chromatin binding agent selected from the group consisting of digoxigenin, a digoxigenin derivative, distamycin and a distamycin derivative. The present invention also relates to a method of producing said improved clot retrieval device. The present invention also relates to a method of prevention and/or treatment of stroke in a subject, comprising removing at least one clot using the clot retrieval device coated with said least one chromatin binding agent.

### CROSS REFERENCE TO RELATED APPLICATION

The present application claims priority of European Patent Application No. 20306701.2 filed Dec. 29, 2020. The entire contents of which are hereby incorporated by reference.

### FIELD OF THE INVENTION

The present invention relates to clot retrieval devices and their therapeutic use.

### BACKGROUND

Acute ischemic strokes (AIS) are the second cause of death in the world according to World Health Organization. AIS caused 6.9 million deaths in 2013 and 3.3 million deaths in 2015, becoming a major public health problem. They are the consequence of a blood stopped-circulation provoked by a thrombus in a brain artery, who leads to a lack of oxygen and nutrients in the ischemic brain area. In fact, the neuronal cells require a continuous oxygenized-blood flow otherwise they die quickly. In order to overturn the ischemic condition, it is necessary to eliminate the thrombus in order to re-establish the blood flow.

To reach this goal, two techniques are used. The first is the thrombolysis who consists in injecting in the blood circulation a drug able to dissolve the thrombus. However, the therapeutic window for this procedure is very tight: the European taskforce recommends proceeding with thrombolysis no later than 4 hours and a half after the first symptoms and in the USA this time falls to 3 hours. The issue is serious since the administration of this treatment beyond the recommended time frame can cause brain hemorrhages, which irremediably aggravates the cerebral tissue damages.

The second technique, developed in 2015, consists in the mechanical retrieval of the occluding thrombus, which is called thrombectomy. Under direct visualization of the arterial lumen, the thrombus is crossed by a guide connected to a stent retriever. The stent is then deployed and pulled; the stent meshes can then trap the thrombus and aid retrieving it throughout the arterial tree. The current indication for this strategy targets large arteries in imaging-visible area and its use is allowed only within 6 hours since the first symptoms. A successful mechanical thrombectomy can help the patients returning to an autonomous life after the intervention.

Nevertheless, surgeons have sometime troubles to pull out the thrombus and sometimes some thrombus pieces cannot be removed.

There is therefore a need for improving efficiency of clot retrieval devices, in particular for increasing the chances of removing the whole thrombus and reducing the risks of mechanical arterial damage.

### DETAILED DESCRIPTION OF THE INVENTION

The Inventors have surprisingly found that coating an agent targeting extracellular chromatin on a clot retrieval device allows improving the capture of a thrombus. Digoxigenin and distamycin were successfully used as extracellular chromatin targeting agent.

Intracerebral thrombi indeed contain a high quantity of genetic material, organized such like a complex mesh crossing the inside of the thrombus as well as covering the thrombus surface. This genetic material probably derives from the process of “NETosis” of the neutrophils trapped inside of the thrombi. The Neutrophil Extracellular Traps (NETs) are released by activated neutrophil and contain several components of chromatin: mainly DNA, but also histones and enzymes that regulate the biology of chromatin, such as topoisomerases. The complex mesh formed by the NETs is thought to play an important role in consolidating the thrombus and anchoring it to the vascular wall of patients with AIS (Acute Ischemic Stroke).

The coating according to the invention thus allows the adhesion of the clot retrieval device to the biologic moiety of the thrombus, thereby improving capture of the whole thrombus. The stable capture of the chromatin can indeed guarantee the retrieval of the thrombus in one whole piece (thus reducing or avoiding its fragmentation along the retrieval pathway of the guide). Besides, the immediate and firm capture of the thrombus allows reducing the number of stent retriever and its delivery system passages through the cerebral arterial tree and hence reducing the risk of mechanical, procedure-related, arterial damage.

Targeting chromatin is particularly advantageous, since chromatin is not be present in the blood flow nor on the vascular wall, thereby avoiding saturation of the clot retrieval device through its journey from the arterial access through to the occlusion site in the blood circulation or its adhesion to the arterial wall at the site of thrombectomy.

Since digoxigenin and distamycin cannot directly adhere to the metallic surface of clot retrieval devices, an intermediate layer of polymer can be used to functionalize the surface of the clot retrieval device and render it suitable to be coated with the chromatin binding agents.

The clot retrieval device coated with a chromatin binding agent thus allows limiting the risk of micro-embolism during the removal of the thrombus as well as risk of mechanical, procedure-related, arterial damage.

A first object of the invention is thus a clot retrieval device, preferably a stent retriever, wherein at least the part of said device intended to be in contact with a clot is coated with at least one chromatin binding agent, wherein said chromatin binding agent is selected from the group consisting of digoxigenin, a digoxigenin derivative, distamycin and a distamycin derivative.

Said chromatin binding agent is preferably coated on the part of the device intended to be in contact with a clot via at least one coating agent.

Said coating agent may be PDA or PDA-PEG bis amine.

The clot retrieval device as defined above is preferably characterized in that at least the part of said device intended to be in contact with a clot is coated with at least one coating agent and said chromatin binding agent is linked to said coating agent.

The digoxigenin derivative may be a compound of the following formula (III)

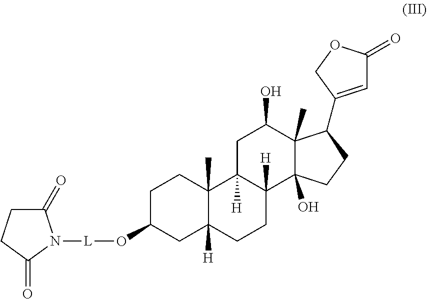

wherein L is a linker comprising at least one group selected from the group consisting of an amino, ester, amido, carboxy and hydroxyl group.

The digoxigenin derivative is preferably a digoxigenin NHS ester.

The distamycin derivative is preferably distamycin azide.

A preferred clot retrieval device as defined above is characterized in that (i) the chromatin binding agent is digoxigenin NHS ester and the coating agent is PDA PEG bis-amine or (ii) the chromatin binding agent is distamycin azide and the coating agent is PDA.

Another object of the invention is a method for producing a clot retrieval device as defined above, wherein said method comprises:
a) coating at least the part of the device intended to be in contact with a clot with at least one coating agent, and
b) contacting the part of the device coated with at least one coating agent obtained in step a) with at least one chromatin binding agent.

Step a) may comprise contacting at least the part of the device intended to be in contact with a clot with a solution comprising dopamine and, optionally, PEG bis-amine.

Another object of the invention is the use of at least one chromatin binding agent for manufacturing a clot retrieval device, wherein said chromatin binding agent is selected from the group consisting of digoxigenin, a digoxigenin derivative, distamycin and a distamycin derivative.

Another object of the invention is a method for removing a clot in a subject, wherein said method comprises removing said clot with a clot retrieval device as defined above or obtained by the method as defined above.

Another object of the invention is a method for preventing and/or treating stroke in a patient in need thereof, wherein said method comprises removing at least one clot with a clot retrieval device as defined above or obtained by the method as defined above.

By “clot retrieval device comprises”, it is herein meant a medical device designed to restore blood flow in patients experiencing ischemic stroke due to vessel occlusion.

Clot and thrombus are herein synonymous.

The clot retrieval device preferably comprises a metallic surface, for example made of at least one transition metal, such as selected from the group consisting of cobalt, chromium, tungsten, nickel, iron, manganese and titanium.

The metallic surface of the clot retrieval device may for example comprise an alloy of at least two transition metals, such as for example an alloy comprising nickel and titanium (such as nitinol) or an alloy comprising cobalt and chromium.

The clot retrieval device is preferably a stent retriever.

A stent retriever is a cylindrical device consisting of a self-expanding stent mounted on a wire and deployed within a catheter.

The clot retrieval device as defined above is coated with at least one chromatin binding agent.

By “chromatin binding agent”, it is herein meant a compound able to bind chromatin.

The skilled person can easily determined if a given compound is able to bind chromatin. For example, the compound to be assessed is coated on a disk, for example a cobalt-chromium disk or a nitinol disk. The disk is washed, for example three times in distilled water, and then immersed in a solution comprising genetic material (in particular genomic DNA and associated proteins), at 37° C. during 5 minutes. Said genetic material is for example obtained from mouse tail, for example using the “F-355L DNA Release” kit (Thermo Scientific). The disk is then thoroughly rinsed to removed unbound genetic material and immersed in a solution containing a DNA labelling marker, for example SYTOX Green, for example during 30 minutes, and washed to removed unbound DNA labelling marker. The signal from the DNA labelling marker on the disk coated with the compound to be assessed is then measured and, for example, is compared to the signal obtained using a non-coated disk. The presence of DNA on the coated disk surface indicates that the assessed compound is able to bind chromatin.

In the frame of the present invention, the chromatin binding agent is selected from the group consisting of digoxigenin, a digoxigenin derivative, distamycin and a distamycin derivative.

The chromatin binding agent may comprise an azide group or a NHS (N-Hydroxysuccinimide) group (also referred to as Su-o).

Digoxigenin is a steroid found in plants, such as Digitalis purpurea, Digitalis orientalis or Digitalis lanata.

Digoxigenin is referred under CAS number 1672-46-4.

Digoxigenin may be obtained by chemical synthesis or by purification from plants.

By “digoxigenin derivative”, it is herein meant a compound derived from digoxigenin, for example by adding at least one functional group to digoxigenin, in particular for improving its linking to a coating agent.

The digoxigenin derivative may thus be obtained by chemical synthesis from digoxigenin.

The digoxigenin derivative preferably comprise a NHS group.

The digoxigenin derivative is for example a compound of the following formula (III):

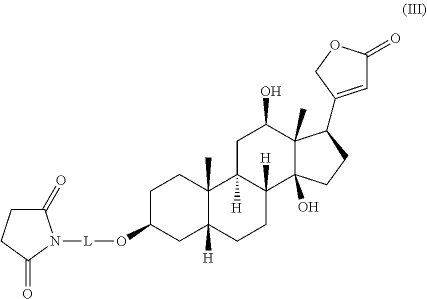

wherein L is a linker, for example a linker comprising at least one group selected from the group consisting of an amino (such as —NH— or —N((C₁-C₁₂)alkyl)-), ester (for example —C(═O)—O— or —O—C(═O)), amido (such as —C(═O)—NH—, —NH—C(═O)—, —C(═O)—N((C₁-C₁₂)alkyl)- or —N((C₁-C₁₂)alkyl)-C(═O)), and hydroxy (—O—) group.

The digoxigenin derivative of formula (III) is for example a compound of the following formula (IV):

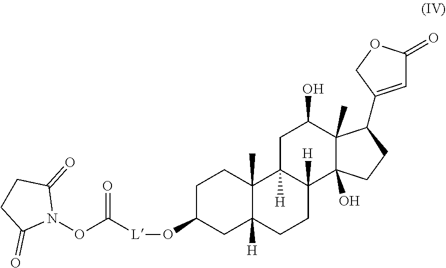

wherein L′ is a linker, for example a linker comprising at least one group selected from the group consisting of an amino, ester, amido, and hydroxy group.

The digoxigenin derivative of formula (III) is preferably a compound of the following formula (V):

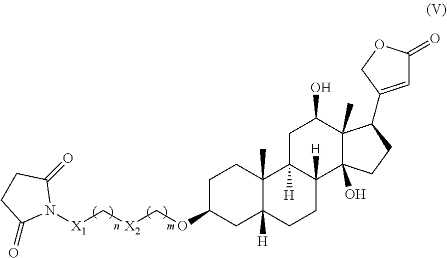

wherein X₁ and X₂ are, independently from each other, one group selected from the group consisting of: —O—, —NH—, —NR—, —O—CO—, —CO—O—, —NR—CO— and —CO—NR—, wherein R represents H or an alkyl comprising from 1 to 12 carbon atoms,
wherein n is an integer comprised from 1 to 10, for example 5, and
wherein m is an integer comprised from 1 to 5, for example 1.

Within the present application, the term “alkyl group” means: a linear or branched, saturated, hydrocarbon-based aliphatic group comprising, unless otherwise mentioned, from 1 to 12 carbon atoms, preferably from 1 to 6 carbon atoms. By way of examples, mention may be made of methyl, ethyl, n-propyl, isopropyl, butyl, isobutyl, tert-butyl or pentyl groups.

In a preferred embodiment, the digoxigenin derivative is a compound of the above formula (V), wherein X₁ is —O—CO—, X₂ is —NH—CO—, n is an integer comprised from 1 to 10, for example 5, and m is an integer comprised from 1 to 5, for example 1.

A preferred digoxigenin derivative as defined above is the compound of the following formula (VI) (also referred to as digoxigenin NHS ester, DIG-NHS or ε-(Digoxigenin-3-0-acetamido)caproic acid N-hydroxysuccinimide ester):

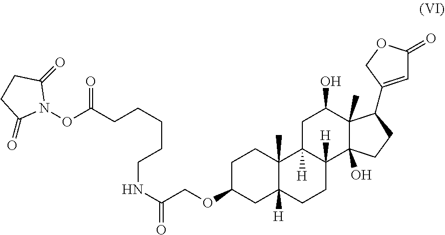

Digoxigenin and digoxigenin NHS ester of are commercially available.

Distamycin (also referred to as Distamycin A) is a pyrrole-amidine antibiotic.

Distamycin A is for example in the form of dystamycin A hydrochloride, which is referred under CAS number 6576-51-8.

Distamycin may be obtained by chemical synthesis.

Distamycin is commercially available.

By “distamycin derivative”, it is herein meant a compound derived from distamycin, for example by adding at least one functional group to digoxigenin, in particular for improving its linking to a coating agent.

The distamycin derivative may thus be obtained by chemical synthesis from distamycin.

The distamycin derivative is for example distamycin azide.

A coating agent is preferably used for coating at least one chromatin binding agent as defined above on the clot retrieval device as defined above.

The coating agent is preferably a polymer or a copolymer able to adhere to a metallic surface.

The coating agent is also a compound safe for use on a medical device.

The coating agent may for example be selected from the group consisting of polydopamine (PDA) and PDA-PEG bis amine (PPba) copolymer.

Polydopamine (PDA) is a synthetic polymer made of L-DOPA (3,4-dihydroxyphenylalanine). PDA is a bio-inspired, self-assembling polymer, which shows exceptional adherence to metallic surfaces.

PDA may be obtained by polymerization of dopamine. Dopamine, a small molecule previously known for its biological role as a neurotransmitter, which combines an amine and a catechol group (which is converted into quinone by oxidation), when dissolved in an aqueous buffer at a slightly basic pH, indeed self-polymerizes into a very adherent film, on various types of substrates.

PDA-PEG bis amine (PPba) is a copolymer obtained by polymerization of dopamine and PEG (poly (ethylene glycol) bis-amine.

The present invention particularly related to a clot retrieval device, wherein at least the part of said device intended to be in contact with a clot is coated with at least one chromatin binding agent.

The clot retrieval device may thus be completely or partially coated with at least one chromatin binding agent.

The “clot retrieval device”, the “chromatin binding agent” and the “coating agent” are particularly as defined above in the sections of the same name.

The chromatin binding agent is thus selected from the group consisting of digoxigenin, a digoxigenin derivative, distamycin and a distamycin derivative.

The part of said device intended to be in contact with a clot is preferably coated with one chromatin binding agent.

Alternatively, the part of said device intended to be in contact with a clot may be coated with two chromatin binding agents or at least two chromatin binding agents.

A preferred chromatin binding agent is digoxigenin, the digoxigenin derivative of formula (III) (preferably the digoxigenin derivative of formula (IV), more preferably the digoxigenin derivative of formula (V), still more preferably digoxigenin NHS ester), distamycin or distamycin azide.

The at least one chromatin binding agent is preferably coated on the part of the device intended to be in contact with a clot via at least one coating agent.

A preferred coating agent is PDA or PDA-PEG bis-amine.

The clot retrieval device as defined above is preferably characterized in that at least the part of said device intended to be in contact with a clot is coated with at least one coating agent and said at least one chromatin binding agent is linked to said coating agent, preferably covalently linked to said coating agent.

The present invention particularly relates to a clot retrieval device as defined above, wherein (i) the chromatin binding agent is digoxigenin NHS ester and the coating agent is PDA-PEG bis-amine or (ii) the chromatin binding agent is distamycin azide and the coating agent is PDA.

The clot retrieval device as defined above may be obtained by the method disclosed below.

The present invention also relates to a method for producing a clot retrieval device as defined above, wherein at least the part of said device intended to be in contact with a clot is coated with at least one chromatin binding agent.

The method particularly comprises:
a) contacting at least the part of the device intended to be in contact with a clot with at least one coating agent, and
b) contacting the part of the device coated with at least one coating agent obtained in step a) with at least one chromatin binding agent.

The “clot retrieval device”, the “chromatin binding agent” and the “coating agent” are preferably as defined above.

The method as define above may comprise a pre-step a) before step a), comprising cleaning the part of the device to be coated with at least one chromatin binding agent, in particular at least the part of the device intended to be in contact with a clot.

This cleaning step may for example comprise:
(i) ultrasonically polishing at least the part of the device intended to be in contact with a clot, preferably in three successive baths (for example acetone, ethanol and finally distilled water), for example 10 minutes for each bath, in order to remove oxidation and organic residues, and/or
(ii) washing at least the part of the device intended to be in contact with a clot in a buffer, preferably a Tris buffer, for example three 3 times, for example during 3 minutes or each wash, preferably under stirring, for example at 400 rpm.

Step a) comprises coating at least the part of the device intended to be in contact with a clot with at least one coating agent.

Step a) thus allows obtaining a clot retrieval device, wherein the surface of at least the part of the device intended to be in contact with a clot is coated with said at least one coating agent. Said surface is also referred to as the “coated surface”.

The coating with the coating agent is preferably obtained by direct polymerization of the coating agent onto the surface of the device.

Step a) as defined above preferably comprises contacting, preferably immersing, at least the part of the device intended to be in contact with a clot with a solution comprising dopamine and, optionally, PEG bis-amine.

Dopamine polymerize to give DPA directly onto the surface of the device.

Dopamine and PEG bis-amine are used for obtaining the copolymer DPA-PEG bis-amine by polymerization directly onto the surface of device.

In a preferred embodiment, step a) may comprise contacting (preferably immersing), preferably under stirring (for example at 400 rpm), the surface of at least the part of the device intended to be in contact with a clot with an alkaline (preferably at pH 8.5) solution comprising dopamine and, optionally PEG bis-amine, preferably in the air and in the dark, preferably at a temperature comprised between 18° C. and 30° C., in particular at room temperature, and preferably for a duration comprised from 15 hours to 30 hours, in particular comprised from 17 h to 24 h.

Step a) preferably further comprises a rinsing step, in particular for stopping the polymerization of the coating agent. Said rinsing step may for example comprise:
washing at least the coated part of the device with water, preferably by at least three washes in distilled water, preferably under stirring, for example during 3 minutes for each wash, and
optionally, a brief sonication of at least the coated part of the device (preferably no more than 15 seconds), after the last wash.

Step a) thus results in a layer of the coating agent onto the surface of at least the part of the device intended to be in contact with a clot.

The layer of coating agent (in particular the PDA layer or the PDA-PEG bis-amine layer) preferably has a thickness comprised from 20 nm to 100 nm, preferably from 30 nm to 50 nm, more preferably of 45 nm.

When the coating agent is PDA, step a) may for example comprise contacting (preferably immersing) at least the part of the device intended to be in contact with a clot with a solution comprising dopamine. Said solution may for example comprise from 0.5 to 10 mg/ml of dopamine, preferably from 1 to 2 mg/ml of dopamine, preferably in a Tris buffer.

When the coating agent is PDA-PEG bis-amine, step a) may for example comprise contacting (preferably immersing) at least the part of the device intended to be in contact with a clot with a solution comprising dopamine and PEG bis-amine, preferably in a weight ratio dopamine:PEG bis-amine from 1:2 to 1:4, for example of 1:3. Said solution may for example comprise from 0.5 to 10 mg/ml of dopamine, preferably from 1 to 2 mg/ml of dopamine, more preferably 1 mg/ml of dopamine, and from 0.5 to 15 mg/ml of PEG bis-amine, preferably from 1 to 6 mg/ml of PEG bis-amine, more preferably 3 mg/ml of PEG bis-amine, preferably in a Tris buffer.

Step b) comprises contacting the part of the device coated with at least one coating agent obtained in step a) with at least one chromatin binding agent.

Step b) allows obtaining a device, wherein at least the part of the device intended to be in contact with a clot is coated with said at least one chromatin binding agent, by linking said at least one chromatin agent to said at least one coating agent, preferably by covalent linking.

Step b) may for example comprise contacting (preferably immersing) the part of the device coated with at least one coating agent obtained in step a) with a solution comprising at least one chromatin binding agent.

The linking between the chromatin agent and the coating agent may be direct or indirect.

By the expression “direct linking”, it is herein meant that the chromatin binding agent is directly linked to the coating agent. This is for example the case when PDA-PEG bis-amine is used as coating agent and digoxigenin NHS ester as chromatin binding agent.

The part of the device coated with at least one coating agent obtained in step a) is for example contacted, preferably immersed, in a solution comprising the chromatin binding agent, for example during 30 minutes, preferably under stirring (for example at 400 rpm), at room temperature and in the dark. The solution comprising the chromatin binding agent may for example be a solution of PBS 1× or sodium bicarbonate (10 mM, pH 8.3).

For example, the part of the device coated with PDA-PEG bis-amine obtained in step a) is immersed in a PBS 1× or sodium bicarbonate (10 mM, pH 8.3) solution comprising from 80 μg/mL to 200 μg/mL of digoxigenin NHS ester, for example from 100 μg/ml to 150 μg/ml of digoxigenin NHS ester.

By the expression “indirect linking”, it is herein meant that the chromatin binding agent is linked to the coating agent via a linker.

The surface of the device coated with the coating agent in step a), in particular the PDA coated surface, is for example modified through the fixation of a biorthogonal, copper-free click chemistry-suitable linker and the chromatin binding agent is linked to the linker by a copper-free click chemistry reaction.

Step b) may thus for example comprise:
contacting, preferably immersing, the part of the device coated with at least one coating agent obtained in step a) with a solution comprising the linker, preferably under stirring, preferably at a temperature comprised between 18° C. and 30° C., in particular at room temperature, and preferably for a duration comprised between 18 hours and 30 hours, in particular comprised between 20 h and 24 h, to obtain a coated surface linked to a linker,
optionally, rinsing the coated surface linked to a linker, in particular with deionized water, and
contacting, preferably immersing, the coated surface linked to a linker with a solution comprising at least one chromatin binding agent, wherein said chromatin binding agent comprises an azide terminal group, preferably in water, preferably at room temperature, preferably during 24 hours, for example at a concentration of the chromatin binding agent of from 0.001 μM/cm² and 200 μM/cm² of surface of the device.

The layer made of the linker may for example have a thickness comprised from 0.03 nm to 3 nm, preferably from 0.1 nm to 0.2 nm, more preferably of 0.15 nm.

The thickness of the layers made of the linker and of the chromatin-binding agent is preferably comprised from 0.5 nm to 15 nm, preferably from 1 nm to 10 nm, more preferably is of 5.6 nm.

The thickness of the coating agent layer and of the layers made of the linker and of the chromatin binding agent is preferably comprised from 20 nm to 200 nm, preferably from 10 nm to 160 nm, more preferably from 30 nm to 120 nm.

The linker is preferably an alkyne derivative, preferably a cyclooctyne derivative. The linker is thus preferably characterized by the presence of at least one triple bond, especially able to react with an azide group, in particular by click chemistry.

According to one embodiment, the linker has the formula (I-1):

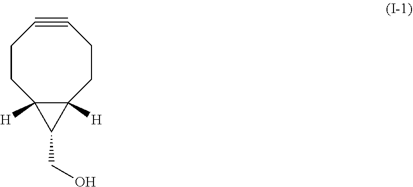

wherein R is a radical of formula —X₁-A₁-NH₂,
   X₁ being chosen from the group consisting of: —CONH—, —CO—, —CS— and —CSNH, X₁ being preferably —CONH— or —CO—, and
   A₁ being an alkylene radical comprising from 2 to 40 carbon atoms, possibly interrupted by at least one oxygen atom.

According to a preferred embodiment, the linker comprising at least one alkyne function has the following formula (I):

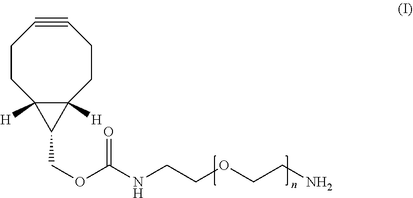

wherein n is an integer comprised between 2 and 14.

Preferably, the linker according to the invention has the following formula (II):

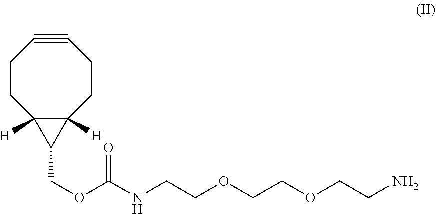 [SMILES: NCCOCCOCCNC(=O)OC[C@@H]1[C@@H]2CCC#CCC[C@@H]21]

Another preferred linker is DBCO (dibenzocyclooctyne)-PEG-amine, for example DBCO-PEG4-amine (also referred under CAS number 1255942-08-5).

The present invention also relates to the use of at least one chromatin binding agent for manufacturing a clot retrieval device, in particular a clot retrieval device having an improve efficacy and safety, wherein said chromatin binding agent is selected from the group consisting of digoxigenin, a digoxigenin derivative, distamycin and a distamycin derivative.

The present invention also relates to the use, in particularly the in vitro use, of at least one chromatin binding agent to improve efficacy and safety of a clot retrieval device, wherein said chromatin binding agent is selected from the group consisting of digoxigenin, a digoxigenin derivative, distamycin and a distamycin derivative.

The “chromatin binding agent” and the “clot retrieval device” are particularly as defined above.

The chromatin binding agent is preferably coated on the surface of at least the part of the clot retrieval device intended to be in contact with a clot, preferably via a coating agent as defined above.

The present invention also relates to a method for removing a clot in a subject in need thereof, wherein said method comprises removing said clot with a clot retrieval device as defined above or obtained by the method as defined above.

The present invention also relates to a method for preventing and/or treating stroke in a subject in need thereof, wherein said method comprises removing at least one clot with a clot retrieval device as defined above or obtained by the method as defined above.

The subject is preferably a mammalian, more preferably a human being.

The invention will be further illustrated by the following figures and examples. However, these examples and figures should not be interpreted in any way as limiting the scope of the present invention.

### BRIEF DESCRIPTION OF THE DRAWINGS

FIG. 1: Experimental plan for assessing chromatin binding

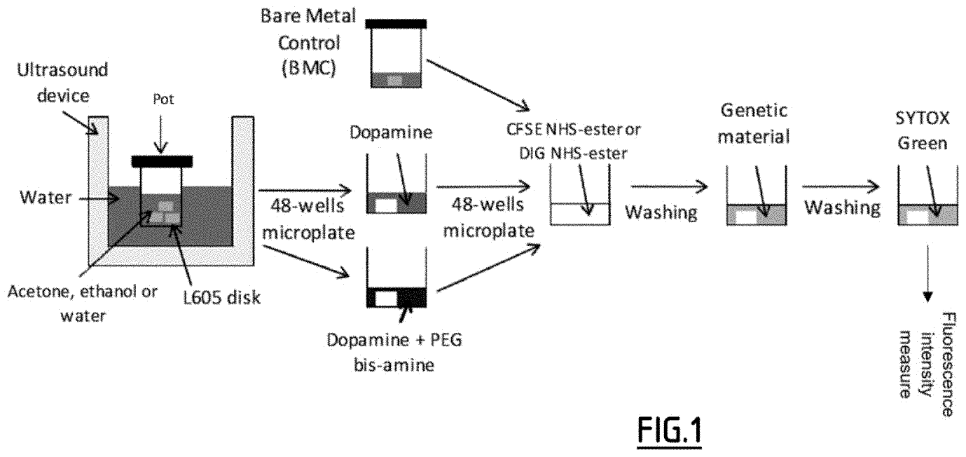

FIG. 2: Optimization of the copolymer for CFSE-NHS ester binding. Different polymerization time (17 or 40 hours), PDA, and PEGba concentration (mg/ml) were evaluated. Each dot represents an individual sample. Effective binding of the CFSE-NHF ester was measured by the peak±SD fluorescence on each sample in the different conditions, both at 17 h and 40 h of polymerization. *PEGba was used after (not together with) PDA). Ordinate: Fluorescence intensity peak value (A.U.).

FIGS. 3A-3B: Digital images of fluorescence intensity. FIG. 3A: Fluorescence intensity of DIG NHS ester at 125 μg/mL in PBS 1× and in sodium bicarbonate; FIG. 3B: The higher fluorescence intensities are both for sodium bicarbonate and PBS 1× at 125 μg/mL. Fluorescence intensities show PBS 1× has a higher fluorescence but a wider peak than sodium bicarbonate.

FIG. 4: Fluorescence intensity of BMC, PDA2, PPba13 and their respective negative controls. PPba13 disks have high fluorescence intensities values with peaks, whereas PDA2 negative control and PDA2 disk are spread along the X-axis. BMC negative control and BMC disks have narrow peaks with fewer fluorescence intensities. Ordinate: Mediane Leverage Residuals

FIGS. 5A-5C: Analysis of DNA binding by Sytox green. FIG. 5A: analysis of the signal homogeneity, via the comparison of the different fields (“champs”). FIG. 5B: analysis of DNA binding extent among groups. FIG. 5C Intersection of the intersection of the two variables (field and group effect). BMC values are in red, PDA values are in blue, and PPba values are in green. There is no champ effect, but a group effect, and no interdependency between champs and groups.

FIG. 6: Median fluorescent intensity of BMC negative control, BMC, PDA2 and PPba13 samples. The median fluorescence intensity of PPba13 sample is higher to the other samples.

FIGS. 7A-7D: Medians comparison for negative controls. FIG. 7(A): One-Way ANOVA analysis FIGS. 7B, 7C and 7D: Champ effect FIG. 7B, group effect FIG. 7C and champs x groups FIG. 7D for signal/noise ratio (Leverage residuals) in ordinate. BMC are represented in red, PPba13 in green, and PDA2 in blue. The PDA2 background noise is significantly higher to the others sets. There is only a group effect. (Ordinate for A: median)

FIG. 8: Enhanced binding properties exerted by Distamycin-coated disks on thrombus-associated NETs. Each dot represents an independent test disk.

### EXAMPLES

### Materials and Methods

### Materials and Chemicals

Cobalt-chromium (L605) disks (4.8 mm diameter, 0.25 mm height, polished on one side) were obtained from Goodfellow (Lille, France). Their composition is described below (Table 1).

TABLE 1
L605 cobalt-chromium chemical composition

| Element | Co | Cr | W | Ni | Fe | Mn |
|---|---|---|---|---|---|---|
| Chemical | 50 | 20 | 15 | 10 | 3.0 | 2.0 |
| composition (% m) | | | | | | |

Tris(hydroxymethyl)aminomethane (Tris) buffer (CAS No. 77-86-1, 10 mM, pH adjusted to 8,5) and Sodium bicarbonate buffer (CAS No. 144-55-8, 100 mM, pH=8,3) were filtered with a 250 mL sterile filter system (polyether sulfone membrane, 0,22 μm pore size, Corning) prior to their use. Ethanol (96% pure) and acetone (99,8%, AnalaR NORMAPUR®) were purchased from VWR Prolabo Chemicals. Dopamine powder (Dopamine hypochloride, Alfa Aesar, A11136, 99% pure), ε-(Digoxigenin-3-0-acetamido)caproic acid N-hydroxysuccinimide ester (DIG NHS ester, 55865-5MG-F), polyethylene glycol bis-amine (PEG bis-amine, P9906) were purchased from Sigma-Aldrich. Antifading agent (Pro Long™ Gold), carboxyfluorescein succinimidyl ester (CFSE, C34554), and SYTOX Green (S7020) were purchased from Thermo Fischer Scientific.

### Determination of an Optimal Copolymer for L605 Alloy Coating

(i) Determination of the copolymer optimal initial concentration of dopamine

### Disks Preparation

Each disk was marked with a diamond point to identify the face in contact with the well bottom at each step. The marked disks were ultrasonically polished (VWR, Ultrasonic Cleaner) in three successive baths (acetone, ethanol and finally distilled water), 10 minutes for each bath, in order to remove oxidation and organic residues. Next the disks were separated in 3 groups: untouched (Bare Metal Controls, BMC), coated with polydopamine (PDA Controls, PDAc) and copolymers samples (PDA+PEG bis amine, PPba). After the polishing step, BMC were placed in distilled and filtered water and left under a sterile culture hood, to avoid contamination. The other disks were washed 3 times in Tris during 3 minutes under stirring onto an orbital shaker (Grant-Bio, PMS-1000i) at 400 rpm.

### Surface Modification by Coating

Disks in the PDAc group were immersed in dopamine only solution (1 or 2 mg/ml) in Tris buffer, whereas PPba disks were immersed in a mixture of dopamine (fixed concentration: 1 mg/mL) and PEG bis-amine solution (concentration varying from 1 to 6 mg/mL) in Tris buffer. Polymerization was allowed by incubation of individual disks in the wells of a sterile and transparent polystyrene 48-wells microplate (under stirring at 400 rpm, in the dark, at room temperature) during 17 hours. Polymerization was stopped by repeated washes in clean distilled water under stirring (400 rpm, 3 minutes each), and a brief sonication (limited to 15 seconds to avoid damaging the coatings), after the last wash.

### Analysis of NHS-Ester Binding by CFSE

For each group (BMC, PDAc, PPba), 3 disks were immersed in a CFSE solution (50 μg/mL in sodium boicarbonate) for 30 minutes at room temperature under stirring (400 rpm), in the dark. The reaction was stopped by washing two times with distilled water (5 minutes each, under stirring at 400 rpm) and the disks were placed, marked face up, onto 20 μL of antifading mounting medium on the bottom of individual wells of 12-wells-Ibidi® slides (Ibidi, Germany) and left untouched for at least 30 minutes in the dark (to allow the antifade mounting medium to polymerize and render the observation by inversed fluorescent microscope possible.

### Fluorescence Microscope Analysis

Digital images were acquired on the coated face of each disk in the green fluorescent channel (ex 475/40 em 530/50 nm) of an Axio Observer® microscope (Zeiss, Germany). The time of exposure was set on the BMC sample that had not been contacted with CFSE (background fluorescence). A second background noise control was obtained by the emission in the near infrared light channel (ex 640/30 em 690/50). The fluorescence intensity histograms from both channels were calculated using the proprietary software (ZEN®, Zeiss, Germany) and raw data were exported and further analyzed using the software Microsoft® Excel.
(ii) Evaluation of chromatin adhesive properties of DIG-coated disks by fluorescence microscopy and SYTOX Green

### Determination of Optimal Conditions for Maximal DIG NHS Ester Coating

DIG NHS ester was diluted in cascade by a 1:2 factor, in either PBS 1× or sodium bicarbonate (10 mM, pH 8.3), from 500 μg/mL to 16,625 μg/mL. PPba disks were immersed individual solutions during 30 minutes, under stirring (400 rpm) at room temperature and in the dark, as for the CFSE ester. The reaction was stopped by washing three times with distilled water (5 minutes each) under stirring. DIG that had effectively onto the disks was detected by indirect immunofluorescence, using a biotynilated anti-DIG NHS ester antibody (NBP2-31191B, Novusbio, USA) and streptavidin PE (554061BD Biosciences, USA) as well as by chromogenic development and absorbance detection, using streptavidin-HRP (557630, BD Biosciences, USA) and TMB (T0440, Sigma). Background signal was set on disks that had been contacted with the buffer alone, in the absence of DIG-NHS). Absorbance was analyzed on the supernatant, on a Infinite® 200 Pro plate reader (TECAN, Switzerland) whereas the amount and distribution of PE at the surface of the washed disks was analyzed by fluorescence microscopy on Ibidi 8 plates, as previously described, in the orange channel (ex 545/25, em 605/710 nm).

### Genetic Material Extraction

Genetic material (genomic DNA and associated proteins) derived from laboratory mouse tails and was obtained using the “F-355L DNA Release” kit (Thermo Scientific). Since the binding of digoxigenin to chromatin is thought to occur via its interaction with topoisomerase, we assessed the presence of proteins associated with the extracted DNA, using the kit “Pierce™ BCA Protein Assay Kit” from Thermo Scientific, prior to using this material.

Functional Analysis of DNA Binding onto Coated Disks

Disks from the three groups were coated in optimal DIG-NHS conditions (125 μg/mL in sodium bicarbonate buffer), washed three times in distilled water and immersed in a solution of mouse tail-derived genetic material (diluted 1:10 in PBS 1× corresponding to a final concentration of 87,3 μg/mL total protein, as determined using the Pierce™ BCA protein assay kit, Thermo Scientific). The incubation with genetic material was carried out at 37° C. and lasted 5 minutes (to mimic the time of contact between the stent retrievers and the clot in stroke patients). The disks were then thoroughly rinsed and immersed in SYTOX Green (167 nM in buffer) for 30 minutes and washed three time with distilled water. Finally, the disks were placed, marked face up, onto anti-fading mounting medium at the bottom of individual wells in a 8-wells Ibidi® slide and analyzed in the green channel.

Artificial thrombi were generated using peripheral human whole blood, collected in dry tubes and stimulated with PMA to generate Nets in order to obtain thrombi containing extracellular Chromatin;

Fresh thrombi were retrieved from patients with stroke.

### Results and Discussion

The presence of chromatin was evaluated at the thrombus surface and in its inside (after its sectioning), by epifluorescence microscopy. The analyzed whole thrombus were retrieved from a cerebral artery in stroke patient.

A dense mesh of chromatin was present in all the analyzed fresh thrombi (n=5) (data not shown).

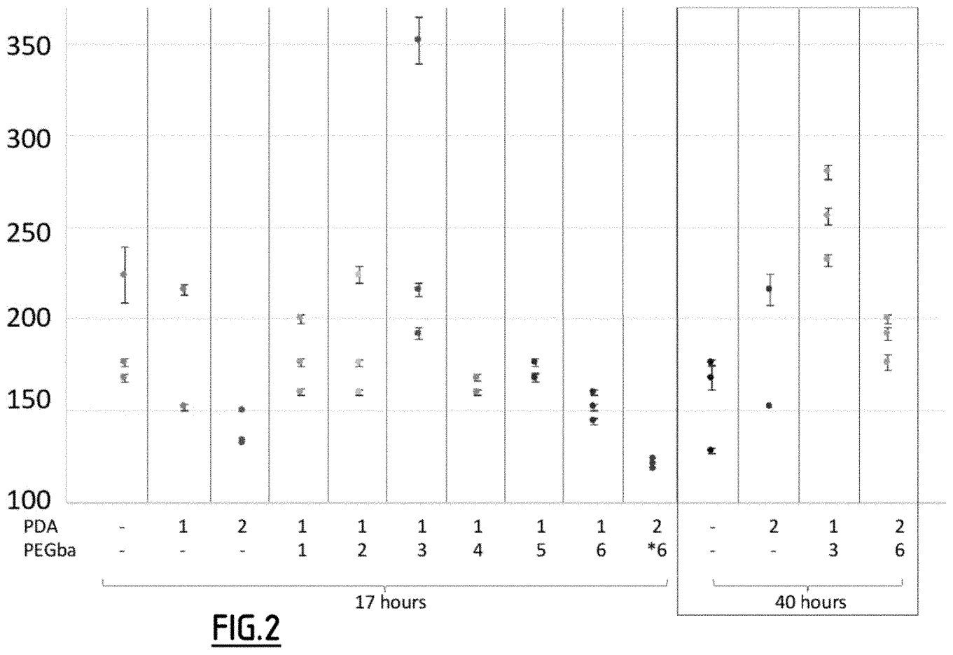

It was first used a two-step dip-coating using PDA in the first bath and PEGba in a subsequent bath. This strategy was intended to orient the grafting of PEGba by one of its amine arms and leave the other amine group available for the subsequent reaction with the NHS ester, in order to drive a unidirectional binding with DIG instead of having disordered bindings. The concentration of PDA at 2 mg/ml was based on the most widely used. The concentration of PEGba was set to 6 mg/mL, to reproduce the ratio 3:1 that had previously been reported as ideal in combination with another self-assembling polymer. Contrary to what expected, the grafting of the CFSE NHS ester was higher onto PDA alone than on PDA+PEGba (see FIG. 2), suggesting that the both of the amines of the PEGba had reacted with the cathecols of the PDA polymer and none was left free and available to react with the NHS ester in the subsequent step.

The strategy was therefore changes and PEGba and PDA were used in the same polymerization bath: the excess in amine groups was supposed to allow the formation of a copolymer with free amines functions at its surface, available for the subsequent reaction with the NHS ester. A copolymer made using the same concentration of the two reagents (2 mg/mL of initial dopamine and 6 mg/mL of PEGba) was compared to PDA alone at 2 mg/ml, BMC, and various concentrations of PEGba used in combination with a fixed concentration of PDA at 1 mg/mL (see FIG. 2). The results show that the use of PDA 1 mg/ml in combination with PEGba 3 mg/ml during 40 h gives the best copolymer condition for subsequent grafting of a NHS ester (CFSE in the case of FIG. 2). This condition has therefore been chosen as the working copolymer solution for the subsequent studies.

Five different concentrations (cascade 1:2 dilutions) of DIG-NHS ester, ranging from 31.25 to 500 μg/ml, prepared in either sodium bicarbonate (100 mM, pH 8.3) or PBS (pH 7.4) were evaluated. The effective grafting of DIG NHS ester onto the PPba copolymer was evaluated using indirect immunofluorescence and an antibody specifically directed against the DIG NHS ester.

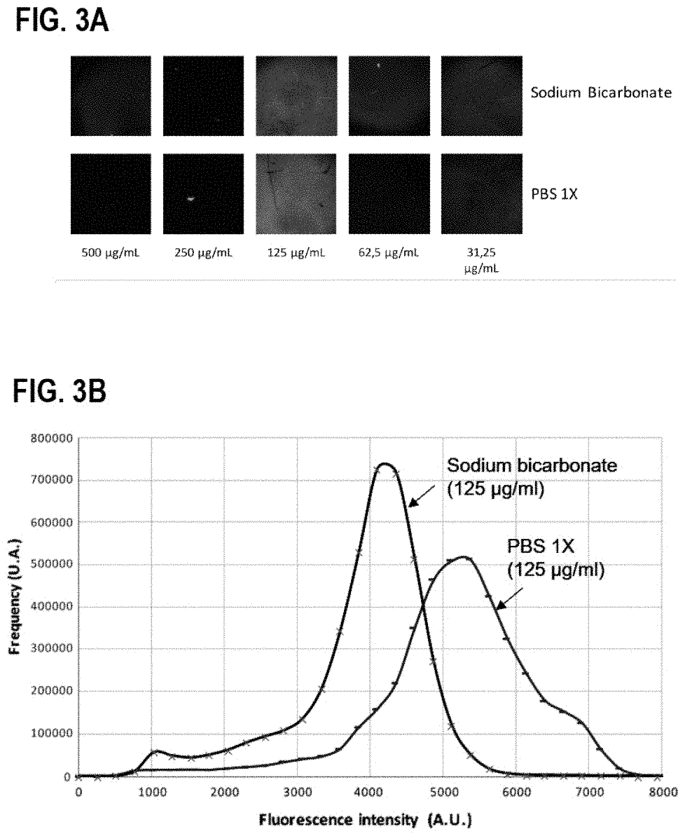

As shown in FIG. 3, the highest disk coverage with DIG, as detected by immunofluorescence intensity, was obtained at the concentration of 125 μg/mL. Both sodium bicarbonate and PBS buffer were effective, but the fluorescence curve obtained with PBS 1×, although yielding a higher peak, was wider than the one obtained with sodium bicarbonate, meaning the density of DIG NHS ester onto the copolymer was more heterogeneous. Sodium bicarbonate and a concentration of 125 μg/ml of DIG NHS was therefore retained for the subsequent functional testing with genetic material.

PPba (1:3) disks were immersed in 125 μg/ml DIG NHS ester solution in bicarbonate sodium buffer, rinsed and contacted during 5 minutes at 37° C. with the genetic material (obtained by extraction form the tail of laboratory mice, diluted in PBS 1×; final protein concentration set to 87,3 μg/mL). The experimental samples were then rinsed and the binding of DNA revealed by SYTOX Green. BMC and PDA-coated disks served as control for specific binding to DIG (vs passive adsorption on the surface).

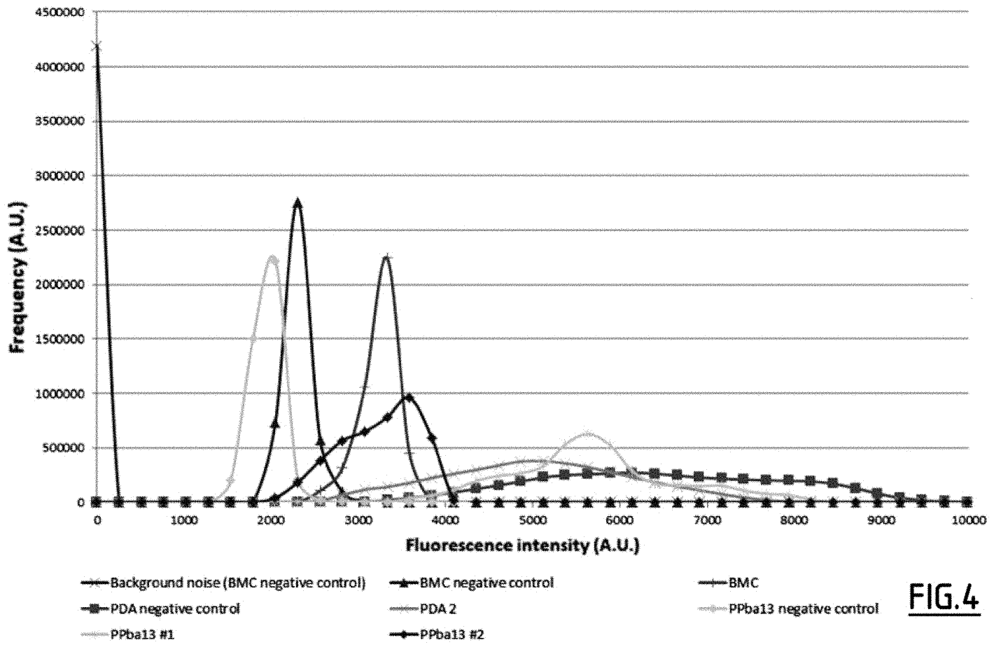

The fluorescence signal obtained at 5× magnification from the different samples was very large (the curves do not display a narrow “peak” but rather a spread along the X-axis) suggesting that the presence of DNA was globally inhomogeneous (see FIG. 4).

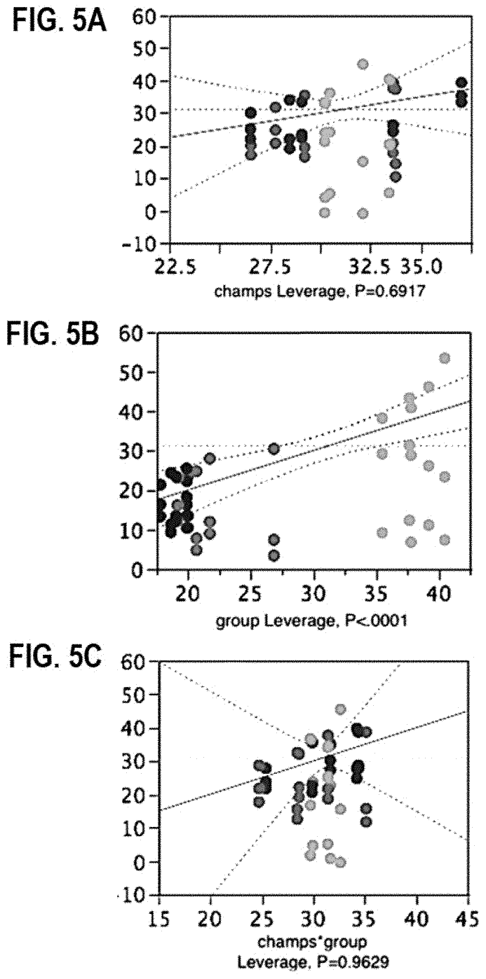

The fluorescence of SYTOX green was assessed at higher magnification (×20) in 5 random fields, on each disk. As shown in FIG. 5, one-way ANOVA analysis ruled out the inhomogeneity of DNA binding as there was no “champs” (field) effect (A, champs leverage, p=NS) in any of the groups (C, champs*group Leverage, P═NS). In contrast, the higher magnification analysis, possibly thanks to the more consistent fluorescence signal, showed that the binding of DNA, as detected by SYTOX green signal, was highest on the disks coated with PPba (green) as compared to those coated with PDA (Blue) or the bare metal controls (BMS Red). These data demonstrate that the affinity binding of DNA through DIG-coated surfaces is consistently superior to what can be achieved with the passive adsorption of genetic material onto the stent-compatible alloy surfaces (panel B, group leverage, p<0.0001).

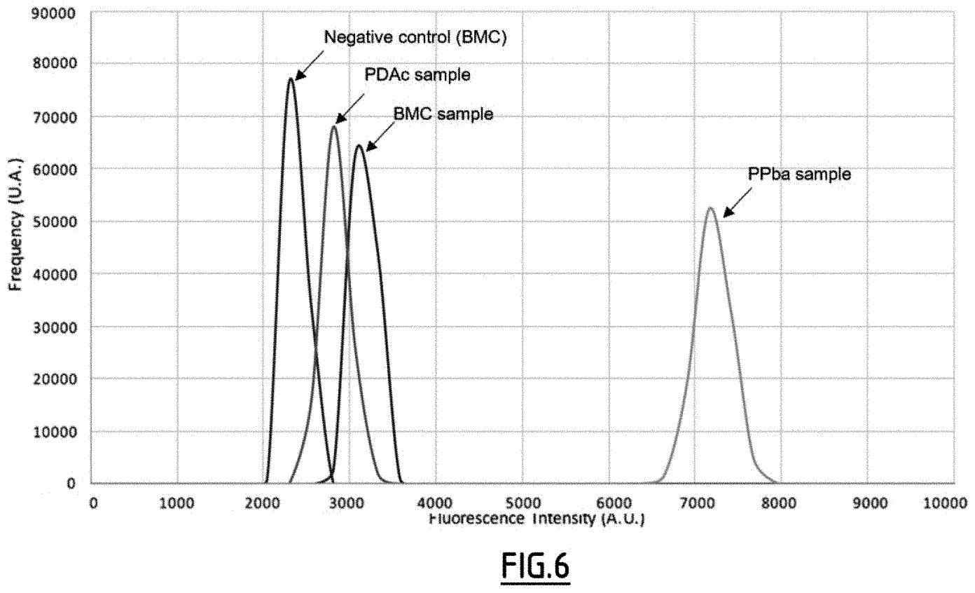

The superiority of functionalized surfaces vs bare metal and control coated surfaces is clear in FIG. 6, which shows a representative fluorescent curve in the SYTOX green channel to compare each group

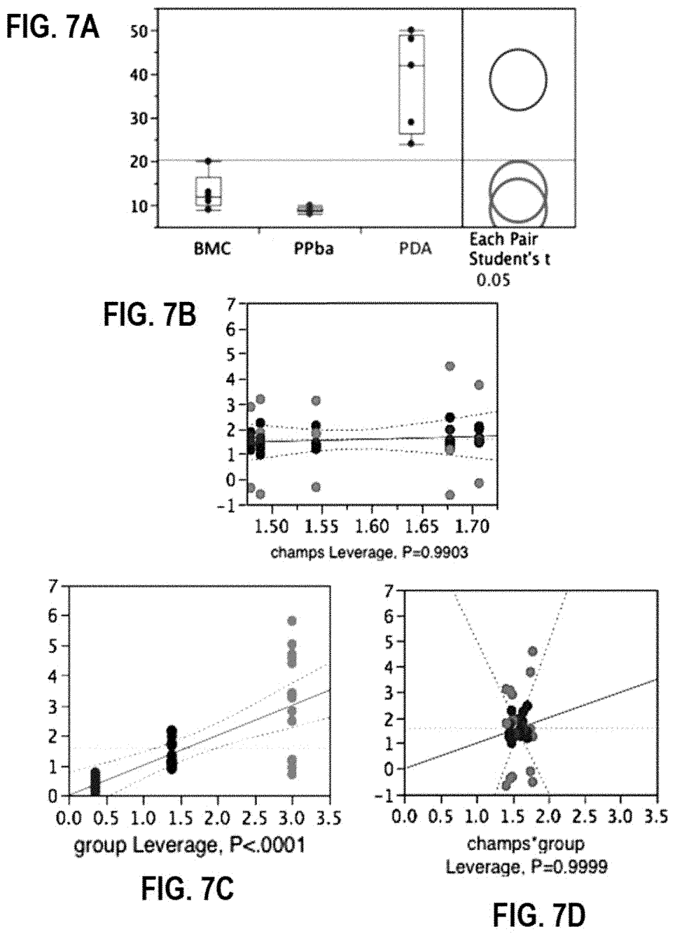

However, the use of fluorescence as a reading out for the functional studies could be biased by the background noise (fluorescence of the negative control in the SYTOX green channel) yielded by the aromatic ring of dopamine, as reflected by the higher background detected in the PDA-coated samples (Median Fluorescence Intensity=38.6 in PDA vs 13 in BMC, FIG. 7 A, p<0.001). However, PDA was also contained in the copolymer but its concentration was reduced by half and this might explain why the median background fluorescence intensity observed in PPba samples is very low and not different from BMC controls (see FIG. 7A). When using the signal/noise ratio (fluorescence in the experimental disks/fluorescence in the control disks) for comparing data between groups, the statistical analysis indicates a group effect (see FIG. 7C, p<0.001) at least as strong as the one observed by comparing absolute signal values from experimental disks (FIG. 5) where the median fluorescence intensity in PDA was likely biased by the background noise (contrary to BMC and PPba disks).

The protocol used for cobalt-chromimum disked was used for the coating of disks made of nitinol (another stent-suitable alloy) and the performance of digoxigenin-coated surface in terms of capture of soluble DNA was confirmed (data not shown).

The coating with distamicyn azide has been produced by a copper-free chemistry protocol.

The ability of fragments of clot retrieval devices coated with digoxigenin-NHS vs those coated with Distamicin-Azide vs bare metal disks to capture DNA and protein material from artificial (in vitro) and from patient's (ex-vivo) thrombi was then evaluated.

To this aim, the tested devices (n=>3/group) were inserted manually in fragments of the same thrombus and hold still within a 3D printed recipient immersed in human plasma (5 minutes, at 37° C.). The tested devices were then removed manually, incubated with DAPI 1 μg/ml and Evan's Blue 1% in PBS for 5 minutes. After careful washing with PBS, the tested devices were mounted using Prolong Gold anti-fade mounting medium in glass bottom dishes and the presence of thrombus captured at their surface was examined by fluorescence microscopy.

Digoxigenin and Distamycin coated devices appeared to be effective for improving the capture of thrombus material as compared with uncoated (bare metal) devices (data not shown). The experiments have been repeated at least three times, yielding similar results (qualitative analysis only).

Nitinol disks were dip-coated in three serial baths composed of
1. Polydopamine (1 mg/ml in tris buffer, pH 8.5, 22±2 hours)
2. DBCO-PEG 4-Amine
3. Azide-derivatized Distamycin or a control irrelevant compound (Control azide).

A fresh thrombus was prepared with human whole blood added to PMA 100 nM and ionomycin 1 μM to promote strong leukocyte activation leading to the release of extracellular chromatin within the thrombus.

Slice of the same thrombus were layered onto coated and bare metal disks in the wells of a 48-well plate, covered with human plasma and incubated at 37° C. on an orbital shaker for 5 minutes.

The thrombus slices were mechanically removed using tweezers, the disk washed in PBS and stained with a solution made of Hoechst 33342 (1 μg/ml, to stain DNA) in PBS for 5 minutes. After careful washing, the presence of thrombus associated—extracellular DNA (NETs) was revealed by fluorescence microscopy in the blue channel.

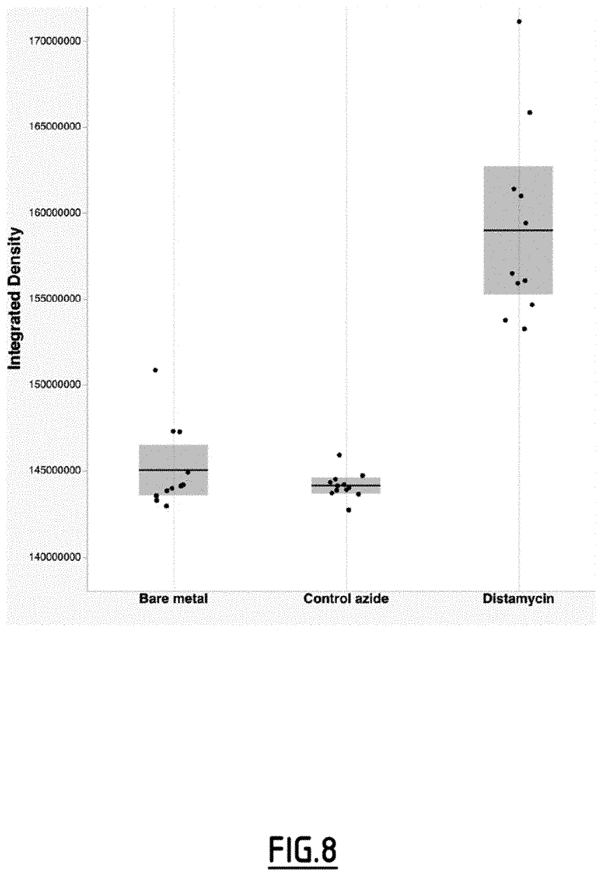

As shown in FIG. 8, distamycin-coated disks have enhanced binding properties on thrombus-associated NETs, by comparison to control conditions (bare metal or control azide).

A coating comprising digoxigenin or distamycin specifically confers the ability to bind genetic material onto stent-compatible metal surfaces. Distamycin and Digoxigenin coated struts both improve the capture of patient's thrombus material as compared with uncoated struts. This bioactive coating is thus suitable for improving the performances of clot retrieval devices.

Besides, the material used for the coating is natural, biocompatible and easily available, and is thus suitable for production at the industrial scale.

1. A clot retrieval device, wherein at least the part of said device intended to be in contact with a clot, comprising a coating comprising at least one chromatin binding agent, wherein said chromatin binding agent is selected from the group consisting of digoxigenin, a digoxigenin derivative, distamycin and a distamycin derivative.

2. The clot retrieval device according to claim 1, wherein said clot retrieval device is a stent retriever.

3. The clot retrieval device according to claim 1, wherein said chromatin binding agent is coated on the part of the device intended to be in contact with a clot via at least one coating agent.

4. The clot retrieval device according to claim 3, wherein said coating agent is PDA or PDA-PEG bis amine.

5. The clot retrieval device according to claim 3, wherein at least the part of said device intended to be in contact with a clot is coated with at least one coating agent and said chromatin binding agent is linked to said coating agent.

6. The clot retrieval device according to claim 1, wherein the digoxigenin derivative is a compound of the following formula (III) 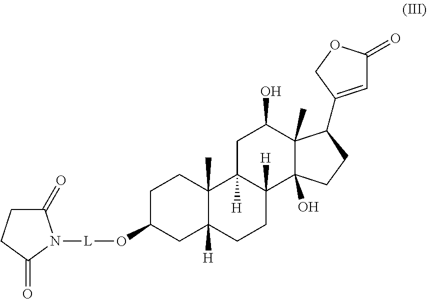
wherein L is a linker comprising at least one group selected from the group consisting of an amino, ester, amido, carboxy and hydroxyl group.

7. The clot retrieval device according to claim 1, wherein the digoxigenin derivative is digoxigenin NHS ester and/or the distamycin derivative is distamycin azide.

8. The clot retrieval device according to claim 3, wherein (i) the chromatin binding agent is digoxigenin NHS ester and the coating agent is PDA PEG bis-amine or (ii) the chromatin binding agent is distamycin azide and the coating agent is PDA.

9. A method for producing a clot retrieval device according to claim 1, wherein said method comprises:
a) coating at least the part of the device intended to be in contact with a clot with at least one coating agent, and
b) contacting the part of the device coated with at least one coating agent obtained in step a) with at least one chromatin binding agent.

10. The method according to claim 9, wherein step a) comprises contacting at least the part of the device intended to be in contact with a clot with a solution comprising dopamine and, optionally, PEG bis-amine.

11. A method for removing a clot in a subject, wherein said method comprises removing said clot with a clot retrieval device according to claim 1 or obtained by the method according to claim 9.

12. A method for preventing and/or treating stroke in a patient in need thereof, wherein said method comprises removing at least one clot with a clot retrieval device according to claim 1 or obtained by the method according to claim 9.
